(19) United States
(12) Patent Application Publication
(10) Pub. No.: US 2006/0147503 A1
(43) Pub. Date: Jul. 6, 2006

(54) Oil absorbent wipe with high crumpability

(75) Inventor: Robert M. Floyd, Maplewood, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 11/026,853

(22) Filed: Dec. 30, 2004

(51) Int. Cl. A61K 9/70

(52) U.S. Cl. 424/443

(57) ABSTRACT

There is provided an oil absorbing wipe material suitable for wiping a users' skin or hair and a method for their manufacture. The wipes comprise at least an oil absorbing porous coextruded film of a crystalline thermoplastic material with a solid diluant containing crumpable layer and a generally nontacky layer. Generally, the wipe changes transparency or color when loaded with oil to provide an oil absorption indication functionality. The wipe is capable of forming a compact ball by crumpling a 10 cm by 10 cm sample to a diameter of 3.0 cm or less.

### BACKGROUND OF THE INVENTION

This invention relates to oil absorbent skin wipe products. The invention particularly relates to oil absorbent skin wiping products with an oil absorption indication function.

A significant amount of oil continuously oozes out of the skin of the face, particularly the nose, cheek and forehead. To maintain cleanliness, reduce shine and to improve the spreadability of cosmetics and other skin products it is important to remove any excess surface oil or sebum. Soap and water work to some extent but there are always times when one is not able to wash. Dry methods of removing these facial oils include the use of thin oil absorbent wipe materials. Oil absorbing wipes for removing facial oil have been described in the art. These wipes generally must be thin, conformable and non-abrasive, considerations not relevant to industrial oil absorbent materials. It is also important that these wipes have the ability to crumple for ease of disposal following use. Generally, the user will crumple the wipe in his or her hand and dispose of it. It is much more difficult to dispose of the wipes if they do not crumple. Paper wipes generally have the ability to crumple, but are deficient in oil removal properties.

Conventional paper type wipes have been used to remove facial oil. For example, natural or synthetic papers using vegetable fibers, synthetic pulp or kenaf have been used. These oil absorbent papers however are often irritating to the skin due to the hard and stiff nature of the fibers. To improve their smoothness, these papers have been continuously calendered and/or coated with powders such as calcium carbonate and sizing agents. Calendering however is not necessarily permanent and surface fibers can reform into a rough surface unless substantial amounts of binder or sizing agents are used, which decreases oil absorption. Paper wipes are also poor indicators as to their effectiveness, as papers generally do not significantly change appearance when they have absorbed oil or sebum.

Improvements to oil absorbing papers are described in Japanese Kokai No. 4-45591 which teaches adhering porous spherical beads onto the surface of an oil absorbing paper so as to solve the problems caused by calendering or coating of paper with powders such as calcium carbonate powders. These beads also are used to allegedly increase the capacity of the papers to absorb sebum. Japanese Unexamined Patent Publication (Kokai) No. 6-319664 discloses a high-density oil absorbing paper produced by mixing (a) a pulp material containing vegetable fibers, as the main component with (b) an inorganic filler, followed by paper-making to form a paper with a basis weight of 0.7 g/cm² or more. However, the oil absorbing papers disclosed in these patent publications still have a limited capacity to absorb oil or sebum and little indicating function as there is little change in opacity or color in the paper when oil is absorbed. Difficulty in confirming oil removal means that users of the oil clearing paper can not evaluate if or how much sebum is removed from the users' face when using the oil absorbing paper such that makeup and the like can be applied with confidence.

An oil absorbing paper for sebum is also disclosed in Japanese Examined Patent Publication (Kokoku) No. 56-8606, or U.S. Pat. No. 4,643,939, which describes a cosmetic oil absorbing paper produced by mixing hemp fibers with 10 to 70% by weight of polyolefin resin fibers and making a paper with a basis weight of from 12 to 50 g/cm². This paper will allegedly clear upon absorption of oil but still requires conventional papermaking techniques and would be rough to the touch. Japanese Unexamined Utility Model Publication (Kokai) No. 5-18392, discloses an oil absorbing synthetic paper comprising an oil absorbing paper with a smooth surface coating of inorganic or organic powder material such as clay particles, silica fine-particles, and powdered fibers. These oil-absorbing papers allegedly have some oil indicating effect by clarifying the paper upon oil absorption thus confirming oil absorption. However, the powder coating lowers the oil absorption capacity for these papers and it is still difficult to attain a clear change in the appearance of this type of oil clearing paper after oil absorption.

Japanese Unexamined Patent Publication (Kokai) No. 9-335451 (WO99/29220) discloses an oil wipe made of a porous thermoplastic film. This oil absorbing wipe film has higher oil absorption capacity than the oil absorbing papers and is also superior in confirming removal of oil following wiping as compared to oil absorbing papers. It is believed that the reason for this good oil removal indicating functionality is that these porous thermoplastic films exhibit low light transmittance before oil absorption because of irregular reflection of light, but the light transmittance increases substantially after the micro-pores of the film are filled with oils producing a large change in the film's opacity or light transmittance, and therefore appearance. This change in opacity clearly confirms to the user the removal of oil or sebum from his or her skin. Further, unlike the paper products, these film based wipes are soft, comfortable, smooth and nonirritating to the skin.

U.S. Patent Application 2004/0121142 describes an oil absorbing wipe having a clear oil indicating function such as described in WO99/29220, which product can readily crumple for disposability and is easy to manufacture, however this was obtained by utilizing high concentrations of a liquid diluent, namely mineral oil, which tended to make the wipe tacky and difficult to dispense from a package of wipes.

### BRIEF SUMMARY OF THE INVENTION

The invention is directed to oil absorbing wipe materials suitable for wiping a users' skin or hair. The wipes comprise at least an oil absorbing porous coextruded film of a crystalline thermoplastic material with a solid diluent containing a crumpable layer and a generally nontacky layer. Generally, the wipe changes transparency or color when loaded with oil to provide an oil absorption indication functionality. The wipe is capable of forming a compact ball by crumpling to a diameter of 3.0 cm or less.

### BRIEF DESCRIPTION OF THE DRAWINGS

FIG. 1 is a diagrammatical view of an apparatus that may be used in the process of the invention to produce a microporous film according to the invention.

FIG. 2 is a perspective view of a dispensable package of oil absorbing wipes.

### DETAILED DESCRIPTION

The invention is generally directed at oil absorbing wipes capable of being dispensed and a dispensable package of oil absorbing wipes of a thermoplastic microporous material. The individual wipes are in the package in a stacked arrangement.

By stacked it is meant that a face of one wipe will be over all of one face, or a substantial portion of that face, in continuous contact with all, or a substantial portion, of a face of an adjacent wipe in the package. Generally, the package will contain at least 2 or more individual wipes, preferably 10 to 1000.

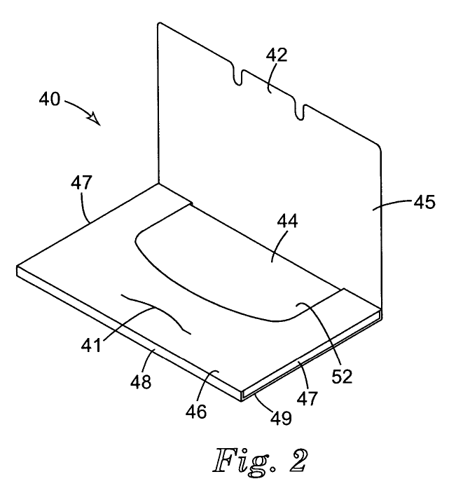

Referring to FIG. 2, a dispensable package of oil wipes in accordance with the invention comprises a dispensable package 40 including individual wipes 44 of oil absorbent wipe material. The package 40 generally comprises a top wall 46 and bottom wall 49, generally parallel to one another, and two side walls 47. A front edge 48 is provided where the back edge is formed into a flap 45, which can be folded down onto the upper face 46 of the package 40. The flap 45 can engage with the package 40 by use of an adhesive or the like, provided as is known in the art. Alternatively, a tab 42 engageable within a slot 41 can be used as a macro-mechanical type closure. Other conventional methods known in the art include the use of cohesive materials, hook and loop fasteners, living hinges, snaps and the like to keep the flap 45 in place to cover the access opening 52 to the wipes. The dispensable package 40 contains an access opening 52, which permits a user to grasp an individual wipe and withdraw it from the package 40 for use. Generally, the access opening 52 is at its largest dimension, smaller than the largest length or width dimension of the dispensable oil absorbing wipe material or wipe.

The oil absorbent wipe is a microporous stretched or oriented coextruded film made of two of more microporous layers of thermoplastic material and a miscible diluent. The microporous thermoplastic material can be coated on at least a portion of one face with an active agent. The wipe, whether used as is or with a coating, is preferably in a dry state, not wet, when used. The coextruded wipe film has a crumpable microporous film layer formed from a solid diluent, and at least one generally nontacky outer layer generally formed using a liquid diluent.

The porosity of the interstitial volume per unit area of the microporous film material layers is preferably in the range of 0.0001-0.005 cm³ as calculated by the equation:
Interstitial volume per unit area=[film thickness (cm)×1 (cm)×1 (cm)×void content (%)]/100 (where the void content is the percentage of voids in the microporous film).

The “void content” is more specifically defined as the percentage of an amount of filling material, when all of the voids of the microporous film are filled with a material of the same composition as the film, with respect to a film with no corresponding voids. The void content of the microporous film is preferably in the range of 5-50% and the thickness is preferably in the range of 5-200 μm.

Various terms are used in the specification and claims herein that may require explanation beyond their generally understood meanings.

Thus, it will be understood that, when referring to the polyolefin polymer or polyolefin-containing polymer as being “crystallized,” this means that it is at least partially crystalline.

It will be further understood that the term “thermoplastic polymer” refers to conventional polymers that are melt processable under ordinary melt processing conditions. The term “thermoplastic polymer” is not intended to include polymers that may be thermoplastic but are rendered melt processable only under extreme conditions.

The term “diluent” refers to a material that (1) is mixable with a polymeric material, (2) is able to form a solution with a polymeric material when the mixture is heated above the melt temperature of the polymeric material, and (3) phase separates from that solution when the solution is cooled below the crystallization temperature of the polymeric material.

The term “solid diluent” refers to a diluent that is solid at room temperature, and solid up to at least about 50° C. That is, the melting temperature of the diluent is above 50° C., and preferably above 60° C.

The term “melting temperature” refers to the temperature at which the material, whether the polymer, diluent, or combination thereof, will melt.

The term “crystallization temperature” refers to the temperature at which the polymer, when present with diluent in the blend, will crystallize.

The term “melting point” refers to the commonly accepted melting temperature of the pure polymer, as may be available in published references.

The crumpable layer of the microporous wipe has the ability to hold a fold, crease, or otherwise crumple into a tight ball. Microporous films made from polymeric material and oil generally are unable to hold a crease or crumple into a ball at typical diluent loading levels. That is, the oil containing microporous materials have a tendency to unfold.

Additionally, crumpable microporous layers are highly diffusive, reflecting visible light at much higher levels than microporous materials made from polymeric material and containing liquid diluent.

The diluent material is miscible with the thermoplastic polymer, preferably a polyolefin, at a temperature above the melting point of the polymer, yet phase separates from the polymer as the polymer crystallizes. When the thermoplastic polymer cools below its crystallization temperature, the polymer regions separate from the diluent to form a material having a continuous polymer phase and a diluent phase. The specific ingredients of the microporous material, as well as methods of making the material will now be discussed in additional detail.

The thermoplastic polymer component of the microporous film layers is preferably a crystallizable polyolefin or polyolefin-containing material. “Polyolefin” refers to a class of thermoplastic polymers derived from olefins, also commonly referred to as alkenes, which are unsaturated aliphatic hydrocarbons having one or more double bonds. Common polyolefins include polyethylene, polypropylene, polybutenes, polyisoprene, and copolymers thereof. “Polyolefin-containing” refers to polyolefin copolymers containing polyolefin or olefin mer units, and mixtures of thermoplastic polymers that include polyolefin. The polyolefin polymer is selected such that it provides good thermally induced phase separation (TIPS) functionality while having suitable properties in the finished film layer, such as strength and handleability.

The microporous layers contain at least about 25 wt-% crystallizable polyolefin-containing polymer, and no more than about 75 wt-%. Typically, the film layers contain about 30 to 70 wt-% polyolefin polymer, and preferably about 35 to 65 wt-% polyolefin polymer. The level of polyolefin in the microporous film layer will largely depend upon the specific polyolefin material used, as will be described in detail below.

Crystallizable thermoplastic polymers suitable for use in a polymer mixture that includes polyolefins are typically melt processable under conventional processing conditions. That is, upon heating, they will easily soften and/or melt to permit processing in conventional equipment, such as an extruder, to form a sheet. Crystallizable polymers, upon cooling under controlled conditions, spontaneously form geometrically regular and ordered crystalline structures. Preferred crystallizable polymers for use in the present invention have a high degree of crystallinity and also possess a tensile strength greater than about 70 kg/cm² or 1000 psi.

Examples of suitable crystallizable thermoplastic polyolefin polymers include polyolefins such as polyethylene (including high-density and low-density), polypropylene, polybutenes, polyisoprene, and copolymers thereof. Many useful polyolefins are polymers of ethylene, but also may include copolymers of ethylene with 1-octene, styrene, and the like.

As mentioned above, the level of polyolefin in the microporous film layers will largely depend upon the specific polyolefin material used. The level of polyolefin will also depend upon the specific diluent material used.

The thermoplastic polymer is combined with one or more diluent compounds to provide the microporous material layers. Diluent compounds suitable for blending with the crystallizable polyolefin-containing polymer to make the microporous oil absorbing wipes of the present invention are materials in which the crystallizable polymer will dissolve or solubilize to form a solution at or above the melting temperature of the crystallizable polymer and the diluent, but will phase separate upon cooling at or below the crystallization temperature of the crystallizable polymer and the diluent.

The diluents that can be used for the outer generally nontacky microporous layer are generally non-volatile hydrocarbon liquids which generally are mixtures of liquids of various molecular weights. Lower molecular weight liquids are generally referred to as light to heavy mineral oils having a carbon chain length of at least about 20. The higher molecular weight liquids, sometimes referred to as semi-solids, are generally more viscous and are referred to as gels such as petroleum jelly or mineral jelly. The semi-solid materials generally have melting points (ASTM D-127) in the range of 30-70° C. The lower molecular weight liquids generally have pour points (ASTM D-97) in the range of 0° C. to -50° C. Examples of preferred nonparticulate fillers that can be used in combination with the aforementioned thermoplastic resins to provide the fine voids include, but are not limited to, mineral oils, petroleum jelly, and mixtures thereof. These nonparticulate fillers are preferred as they exhibit transparency upon absorption of oil. Generally, these fillers are liquids or gels in which the crystallizable polymer will dissolve to form a solution at the melting temperature of the crystallizable polymer, but will phase separate on cooling at or below the crystallization temperature of the crystallizable polymer. Preferably, these nonparticulate fillers have a boiling point at atmospheric pressure at least as high as the melting temperature of the crystallizable polymer. The amount of filler used is preferably in the range of 20-40% by weight, and more preferably 25-40% by weight of the starting thermoplastic material. If the amount of filler added to the starting material is under 20% by weight, the void content of the film layer resulting after stretching is reduced, thus lowering the amount of oil absorption, while if it is above 40% by weight the layer becomes tacky making the wipe difficult to dispense.

The microporous materials of the crumpable layer generally contains a combination of a crystallizable polyolefin polymer and a solid diluent material, which are present during formation of the microporous materials and also present in the microporous materials. The diluent material is solid at room temperature at atmospheric pressure. Most often, the solid diluent is a wax. The term “wax” is applied to a large number of chemically different materials. Waxes are generally solid at room temperature (20° C.) and melt at temperatures greater than about 50° C. Waxes are thermoplastic in nature. In the most general terms, waxes are “naturally” or “synthetically” derived. Natural waxes include animal waxes (such as beeswax, lanolin, tallow), vegetable waxes (such as carnauba, candelilla, and soy), and mineral waxes such as fossil or earth waxes and petroleum (such as paraffin and microcrystalline). Synthetic waxes include ethylenic polymers and copolymers, which include polyethylenes and ethylene-propylene copolymers. These waxes are low molecular weight ethylene homopolymers, and are generally linear and saturated.

Paraffin waxes are derived from the light lubricating oil distillates. Paraffin waxes contain predominantly straight-chain hydrocarbons with an average chain length of 20 to 30 carbon atoms. Paraffin waxes are characterized by a clearly defined crystal structure and have the tendency to be hard and brittle. The melting point of paraffin waxes generally falls between about 50° C. and about 70° C.

Microcrystalline waxes are produced from a combination of heavy lube distillates and residual oils. They differ from paraffin waxes in that they have poorly defined crystalline structure, a generally darker color, and generally higher viscosity and melting points. Microcrystalline waxes tend to vary much more widely than paraffin waxes with regard to physical characteristics. Microcrystalline waxes can range from being soft and tacky to being hard and brittle, depending upon the compositional balance.

Other materials that are not necessarily waxes may also be suitable as solid diluents. For example, suitable solid diluents include low molecular weight polymers or copolymers.

The melting point of the solid diluent material is greater than room temperature, i.e., the melting point is at least about 50° C., so at room temperature (about 20° C.), the diluent is a solid material. The solid diluent is selected, for use with a specific polyolefin polymer, so that the difference in melting points of the two materials is generally at least 25° C. and preferably at least 40° C., although it is understood that materials with lesser melting point differences may be suitable. Typically, the solid diluent will have a melting point that is less than the melting point of the polymer.

Also when selecting a solid diluent for use with a specific polymer, it should be selected so that the polymer is soluble in the melted diluent. However, the polymer should not be so soluble that the melt blend does not hold its shape.

Specific examples of commercially available products that are suitable as solid diluents include paraffin wax under the tradeneme “IGI 1231” from International Group, Inc. (having a melting point of about 53° C.), microcrystalline waxes under the tradenames “Mulitwax W-835” from Crompton-Witco (having a melting point of about 74-80° C.), “Multiwax 180-W” (having a melting point of about 80-87° C.) and “Multiwax W-445” (having a melting point of about 77-82° C.), and low molecular weight polyethylene waxes under the tradename “Polywax 400” (having a melting point of about 81° C.) and “Polywax 500” (having a melting point of about 88° C.), from Baker Petrolite. An alternate term for low molecular weight polyethylene waxes is Fischer-Tropsch waxes, such as available from Sasol. “Sasolwax C80” is similar to Polywax 500. Another commercially available product that is suitable as a solid diluent is short chain ethylene/propylene copolymer under the tradename “EP-700” (having a melting point of about 96° C.) from Baker Petrolite.

As mentioned above, the level of solid diluent in the microporous crumpable film layer will largely depend upon the specific solid diluent material used. The level of solid diluent will also depend upon the specific polyolefin polymer used. Often, a higher molecular weight diluent is present at higher levels than lower molecular weight diluent.

For example, microporous crumpable film layers incorporating high-density polyethylene (HDPE) typically contain 50 to 75 wt-% solid diluent, preferably 60 to 70 wt-% solid diluent, but again, based largely upon the diluent used. For example, when Polywax 400 is used in HDPE, the Polywax 400 is preferably present at a level of at least 55 wt-%, and when Polywax 500 is used, it is present at a level of at least 65 wt-%. When Crompton W-835 microcrystalline wax is used in HDPE, the wax is preferably present at a level of at least 60 wt-%. When IGI 1231 paraffin wax is used in HDPE, the wax is preferably present at a level of at least 60 wt-%.

As another example, microporous crumpable film layers incorporating polypropylene (PP) typically contain 25 to 70 wt-% solid diluent, preferably 35 to 65 wt-% solid diluent, but again, based largely upon the diluent used. For example, for Polywax 400, Polywax 500, and EP-700, the solid diluent is present at a level of at least 35 wt-%, preferably about 35 to 50 wt-%. For IGI 1231 paraffin wax, the wax is preferably present at levels of 35 to 70 wt-%.

And as yet another example, microporous crumpable film layers incorporating methylpentene copolymer (TPX) typically contain 45 to 65 wt-% solid diluent, preferably 55 to 60 wt-% solid diluent, but again, based largely upon the diluent used. For example, when IGI 1231 paraffin wax is used, the wax is present at a level of at least 45 wt-% and is preferably present at a level of 50 to 65 wt-%.

A particular combination of polymer and diluent for either the crumpable or generally nontacky layers may include more than one polymer, i.e., a mixture of two or more polymers and/or more than one diluent.

Nucleating agents are materials that may be added to the polymer melt of the microporous film layers as a foreign body. When the thermnoplastic polymer cools below its crystallization temperature, the loosely coiled polymer chains orient themselves about the foreign body into regions of a three-dimensional crystal pattern to form a material having a continuous polymer phase and a diluent phase.

Nucleating agents work in the presence of melt additives in the thermally induced phase separated system of the present invention. The presence of at least one nucleating agent is advantageous during the crystallization of certain thermoplastic polymeric materials, particularly polyolefins such as polypropylene, by substantially accelerating the crystallization of the polymer over that occurring when no nucleating agent is present. This in turn results in a film with a more uniform, stronger microstructure because of the presence of increased number of reduced-sized domains. The smaller, more uniform microstructure has an increased number of fibrils per unit volume and allows for greater stretchability of the materials so as to provide higher void porosity and greater tensile strength than heretofore achievable. Additional details regarding the use of nucleating agents are discussed, for example, in U.S. Pat. No. 6,632,850 and in U.S. Pat. No. 4,726,989.

The amount of nucleating agent must be sufficient to initiate crystallization of the thermoplastic polymer at enough nucleation sites to create a suitable microporous material. This amount can typically be less than 0.1 wt-% of the diluent/polymer mixture, and even more typically less than 0.05 wt-% of the diluent/polymer mixture. In specific implementations the amount of nucleating agent is about 0.01 wt-% (100 ppm) to 2 wt-% of the diluent/polymer mixture, even more typically from about 0.02 to 1 wt-% of the diluent/polymer mixture.

Useful nucleating agents include, for example, gamma quinacridone, aluminum salt of quinizarin sulphonic acid, dihydroquinoacridin-dione and quinacridin-tetrone, triphenenol ditriazine, two component initiators such as calcium carbonate and organic acids or calcium stearate and pimelic acid, calcium silicate, dicarboxylic acid salts of metals of the Group IIA of the periodic table, delta-quinacridone, diamides of adipic or suberic acids, calcium salts of suberic or pimelic acid, different types of indigosol and cibantine organic pigments, quiancridone quinone, N′,N′-dicyclohexil-2,6-naphthalene dicarboxamide (NJ-Star NU-100, ex New Japan Chemical Co. Ltd.), and antraquinone red, phthalo blue, and bis-azo yellow pigments. Preferred agents include gamma-quinacridone, a calcium salt of suberic acid, a calcium salt of pimelic acid and calcium and barium salts of polycarboxylic acids.

The nucleating agent should be selected based on the thermoplastic polymer being used. The nucleating agent serves the important functions of inducing crystallization of the polymer from the liquid state and enhancing the initiation of polymer crystallization sites so as to speed up the crystallization of the polymer. Thus, the nucleating agent may be a solid at the crystallization temperature of the polymer. Because the nucleating agent increases the rate of crystallization of the polymer by providing nucleation sites, the size of the resultant polymer domains or spherulites is reduced. When the nucleating agent is used to form the microporous film layers, greater amounts of diluent compound can be used relative to the thermoplastic polymer forming the microporous materials.

By including a nucleating agent, the resultant domains of olefin-containing polymer are reduced in size over the size the domains would have if no nucleating agent were used. It will be understood, however, that the domain size obtained will depend upon the additive, component concentrations, and processing conditions used. Because reduction in domain size results in more domains, the number of fibrils per unit volume is also increased. Moreover, after stretching, the length of the fibrils may be increased when a nucleating agent is used than when no nucleating agent is used because of the greater stretchability that can be achieved. Similarly, the tensile strength of the resultant microporous materials can be greatly increased. Hence, by including a nucleating agent, more useful microporous materials can be prepared than when nucleating agents are not present.

Use of a nucleating agent is preferred when using polypropylene polymer, due to the morphological structures formed by polypropylene's inherent crystalline nature during the phase separating process.

Various additional ingredients may be included in the microporous film layers of the present invention wipe product. These ingredients may be added to the polymeric blend melt, may be added to the material after casting, or may be added to the material after stretching of the wipe material, as will be described below.

Most optional ingredients are added to the polymeric blend melt, with the thermoplastic polymer and the diluent, as melt additives. Such melt additives can be surfactants, antistatic agents, ultraviolet radiation absorbers, antioxidants, organic or inorganic colorants, stabilizers, fragrances, plasticizers, anti-microbial agents, flame retardants, and antifouling compounds, for example.

The amounts of these optional ingredients is generally no more than about 15 wt-% of the polymeric blend melt, often no more than 5 wt-%, so long as they do not interfere with nucleation or the phase separation process.

Methods for Making the Microporous Coextruded Film

Production of microporous films requires melt blending a crystallizable thermoplastic polymer and the diluents into two homogenous mixtures or solutions. The polymers are soluble in the liquid diluent or the melted solid diluent. After the materials have been melt blended, they are coextruded, using conventional methods cooled to a temperature at which, for the crumpable layer the solid diluent solidifies and the thermoplastic polymer crystallizes, so as to induce phase separation between the thermoplastic polymer and the solid diluent. An example of a suitable melt mixing method is extrusion, and examples of suitable film forming methods are the blown or tubular film method and the casting method. The blown film method, for example, can give tube-shaped films by melt mixing the main starting material, etc. and then blowing it up from a circular die. The casting method can give films by melt mixing the main starting material, etc. and then extruding it from a die onto a smooth or patterned chilled roll (cold roll). In a modified form of this casting method, the nonparticulate additives and/or fillers may be removed by washing off or extracting with a suitable solvent after extrusion of the melted mixture onto the chilled roll. The melted material may also be filtered when extruded to remove any impurities that might be present.

The polymers in the layers are present as domains of polymer. In some embodiments, these domains are spherulitic or may be spherulites or an agglomerate of spherulites; in other embodiments, the domains may have a “lacey” structure. Adjacent domains of polymer are distinct, but they have a plurality of zones of continuity. There are areas of contact between adjacent polymer domains where there is a continuum of polymer from one domain to the next adjacent domain in such zones of continuity. The polymer domains are generally surrounded or coated by the diluent, but not necessarily completely. Diluent generally occupies at least a portion of the space between domains.

The formed article (before any stretching, which is described below) is generally semi-transparent and/or translucent.

Thereafter the coextruded film is typically stretched in at least one direction to provide a network of interconnected micropores throughout the film. The stretching step generally includes biaxially stretching. The stretching step provides an area increase in the shaped article of from about 10% to over 1200% over the original area of the shaped article. The actual amount of stretching desired will depend upon the particular composition of the film and the degree of porosity desired.

Stretching may be provided by any suitable device, which can provide stretching in at least one direction, and may provide stretching both in that direction and in the other direction. Stretching should be uniform to obtain uniform and controlled porosity. The film is generally first stretched in the web, machine or longitudinal direction, and then in the cross-web or transverse direction.

The microporous films can be dimensionally stabilized according to conventional, well known techniques, such as by heating the stretched sheet, while it is restrained, at a heat stabilizing temperature. Upon stretching, the polymer domains are pulled apart, permanently attenuating the polymer in zones of continuity, thereby forming fibrils and minute voids between diluent coated domains, and creating a network of interconnected micropores. Such permanent attenuation also renders the article opaque, by drastically increasing the diffusing characteristics of the film. A microporous stretched thermoplastic film obtained in this manner has a large percentage of voids constituting the volume of the wipe compared to conventional paper oil cleaning wipes, and has excellent absorption of skin oils per unit area. Also, since the thermoplastic film has a structure with a uniform distribution of many fine voids, prior to wiping of skin oils from the skin surface it appears non-transparent due to light dispersion by the pore structures. However, after oil absorption the oils fill the voids or pores thus either preventing or reducing the degree of light dispersion. This together with the original opaque or transparent nature of the thermoplastic forming the film allows the oil absorbing effect to be clearly assessed by a change in transparency or opacity.

It has been determined that for each polymer melt mixture for the crumpable layer, comprising the polyolefin, solid diluent, and any optional ingredients, an optimum stretch temperature range exists for the first stretching operation. This dictates the optimum stretch for the coextruded film as a whole as the liquid type diluents typically used in the generally nontacky layer is less critical. This optimum stretch temperature is dependent upon the particular polyolefin, the specific solid diluent, and the relative amounts of these components. The optimum stretch temperature can be either above or below the melting point of the solid diluent.

If the material is stretched at this optimum stretch temperature or temperature range, the material becomes opaque and microporous. If stretched either at temperatures above or below the optimum range, full opacity is not obtained; indeed, in some embodiments, the material remains generally transparent and is not microporous. This observed trait is much less apparent when liquid diluents are used; with liquid diluents, the material becomes opaque at a broader range of stretching temperatures. For solid diluent containing systems, these stretching temperature ranges are narrow, often less than about 8° C.

Various examples of stretch temperatures for the crumpable layer are as follows: a microporous material of HDPE and Polywax 400 polyethylene wax has an optimum stretch temperature of about 60° C., whereas HDPE with IGI 1231 paraffin wax has an optimum stretch temperature of about 63° C.; polypropylene (PP) with Polywax 400 has an optimum stretch temperature of about 77° C., and methylpentene copolymer (TPX) with IGI 1231 has an optimum stretch temperature of about 75° C. It is understood that the specific stretch temperatures will vary based on the polymer, diluent and optional ingredients.

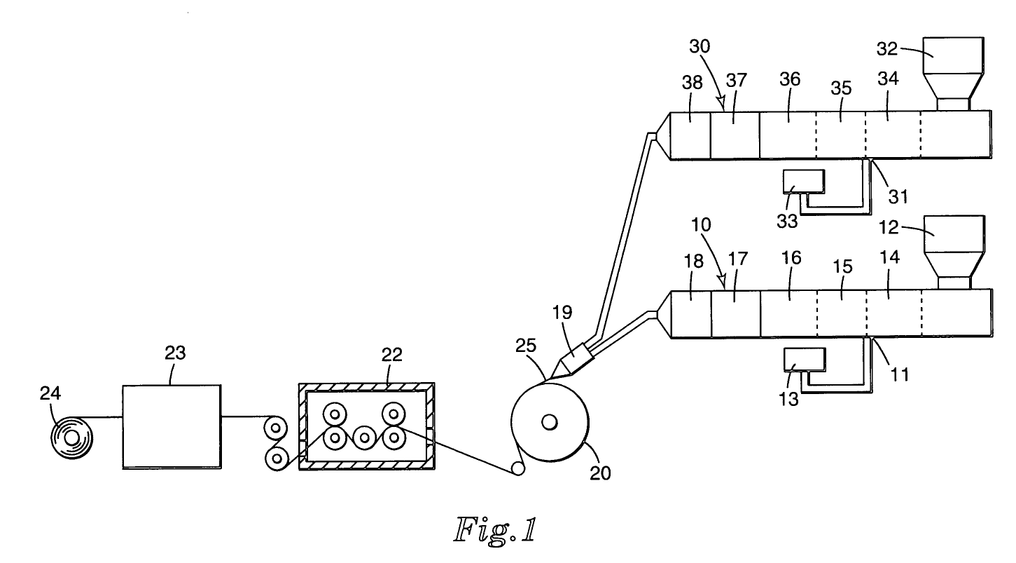

Reference will now be made to the apparatus of FIG. 1 in order to illustrate one preferred method for practicing the present invention. At least two extruder apparatus 10, 30, having hoppers 12, 32 and various zones, is illustrated. Polymer is introduced into hopper 12, 32 of extruder apparatus 10, 30. For extruder 10 solid diluent is melted or softened by device 13 and fed into extruder 10 via a port 11 in the extruder wall between hopper 12 and an extruder exit 17. In other embodiments, port 11 may be positioned proximate hopper 12. For extruder 30 liquid diluent is fed via port 31 prior to the extruder exit 37.

Extruder 10 preferably has at least three zones 14, 15, and 16, which are respectively heated at decreasing temperatures towards extruder exit 17. Extruder 30 also can have zones 34, 35 and 36 for heating leading to the extruder exit 37. The feeds from the two, or more, extruders 10, 30 are converged at the slot die 19. The slot die 19, having a slit gap of about 25 to about 2000 micrometers, is positioned after the extruders.

It is also suitable to include suitable mixing devices such as a static mixers 18, 38 between extruder exits 17, 37 and slot die 19 to facilitate the blending of the polymer/diluent solution. In passing through extruders 10, 30, the mixtures of polymer and diluent is heated to a temperature at or at least about 10° C. above the melting temperature of the melt blend, but below the thermal degradation temperature of the polymers. The mixtures are mixed to form melt blends that are extruded through slot die 19 as a multi layer flowstream 25 onto a quench wheel 20 maintained at a suitable temperature below the crystallization temperature of the polymers and the diluent.

The cooled film may then be led from quench wheel 20 to a machine-direction stretching device 22 and a transverse direction stretching device 23, and then to a take-up roller 24 for winding into a roll. Stretching in two directions as done by the apparatus of FIG. 1 is, of course, optional. The coextruded microporous film is then converted into suitable sized wipes and packaged.

A further method of forming a microporous film from the blended melts includes casting the extruded melts onto a patterned chill roll to provide areas where the blend does not contact the chill roll to provide a membrane of substantially uniform thickness having a patterned surface, the patterned surface providing substantially skinless areas having high microporosity and skinned areas of reduced microporosity. Such a method is described in U.S. Pat. No. 5,120,594 (Mrozinski). The film can then be oriented, i.e., stretched.

The layers of the wipes can be formed of the same or different polymers or polymer blends. Similar blends will provide easier processablity and layer cohesion, however different polymer or blends can be used for aesthetic properties, increasing softness or rigidity or other combinations of properties The crumpability of the wipe is generally 3.0 cm or less and preferably 2.5 cm or less. The overall wipe generally has a thickness of at least 20 microns preferably 35 to 45 microns. The crumpable layer thickness is generally at least about 10 microns or from 10 to 25 microns or 15 to 20 microns where the liquid diluent containing layer is at least about 5 microns or from 5 to 15 microns, where the overall wipe has a Coefficient of Friction of less than 0.75 or less than 0.6.

The average size of the voids formed by stretching of the material forming the wipe is usually preferred to be in the range of 0.1 to 5 μm. If the void size is under 0.1 μm it becomes impossible to rapidly absorb enough skin oil to create a clear change in transparency, while if it is over 5 μm the amount of oil absorption needed to permit a visible change in transparency may be too great.

The interstitial volume per unit area of the microporous stretched thermoplastic film obtained by the stretching process described earlier is preferably in the range of 0.0001-0.005 cm³, and more preferably in the range of 0.0002-0.001 cm³, as calculated by the equation defined above. If the interstitial volume of the film is under 0.001 cm³ it becomes difficult for the user to hold the oil cleaning wipe formed from the film, while if it is over 0.005 cm³ the amount of oil absorption is too great, and it becomes difficult to clearly assess the oil absorbing effect.

If the original opacity is inadequate to produce a significant enough change in opacity, opacifying agents such as silica, talc, calcium carbonate or other like inorganic powders can be used at low levels. Such powders could be coated on the surface of the wipes or incorporated into the film layers. The invention oil absorbent wipes are generally characterized by the ability to change from opaque to translucent after absorbing only a moderate amount of oil, such as would be present on a person's skin (e.g., from 0 to 8 mg/cm²). The oil absorbent wipes are particularly useful as cosmetic wipes as after absorbing skin oil at the levels excreted from common sebaceous glands, they will turn translucent, thus indicating that the undesirable oil has been removed and that makeup or other skin treatments can be applied. The oil absorbing wipe is generally used as a single layer of the microporous coextruded film material but could be laminated to fibrous web materials, or the like.

The individual discrete wipes can be of any suitable size, however, generally for most applications the wipes would have an overall surface area of from 10 to 100 cm², preferably from 20 to 50 cm². As such, the wipes would be of a size suitable for insertion in a package, which could easily be placed in the user's purse or pocket. The material forming the dispensable containers is generally not of importance and can be formed of suitable papers, plastics, paper film laminates and the like. The shape of the wipes is generally rectangular; however, other suitable shapes such as oval, circular or the like can be used.

The oil-absorbing wipes of the invention can contain or be coated with any suitable active or nonactive ingredients or agents. Additional ingredients can comprises a wide range of optional ingredients. Particularly useful are various active ingredients useful for delivering various benefits to the skin or hair during and after oil removal and cleansing.

The coating compositions can also comprise a safe and effective amount of one or more pharmaceutically-acceptable active or skin modifying ingredients thereof. The term “safe and effective amount” as used herein, means an amount of an active ingredient high enough to modify the conditions to be treated or to deliver the desired skin benefit, but low enough to avoid serious side effects, at a reasonable benefit to risk ratio within the scope of sound medical judgment. What is a safe and effective amount of the active ingredient will vary with the specific active ingredient, the ability of the active ingredient to penetrate through the skin, the age, health condition, and skin condition of the user, and other like factors.

### EXAMPLES

This invention is further illustrated by the following examples that are not intended to limit the scope of the invention. In the examples, all parts, ratios and percentages are by weight unless otherwise indicated. The following test methods were used to characterize the microporous films in the examples:

### Test Methods

Gurley Air Flow

This test is a measurement of time in seconds required to pass 50 cm³ of air through a film according to ASTM D-726 Method B.

Porosity

A calculated value based on the measured bulk density of the stretched film and the polymer plus diluent composite density before stretching using the following equation: Porosity=(1−(bulk density/composite density))×100.

Bubble Point Pore Size

Bubble point is the largest pore size in the overall film composite as determined according to ASTM F-316-80. The testing liquid was used to fill the pores of the film. Pressure is applied until flow as bubbles takes place through the largest passageway through the film. The bubbles are observed from a tube that is connected to the low pressure side of the test cell and that is submerged in water. The necessary pressure depends on the surface tension of the test liquid and the size of the largest passageway. Bubble points were determined using Fluoroinert FC-43 liquid (bubble point=6.64/breakthrough pressure in psi or bubble point=4.58×10⁴/pressure in pascals) available from 3M Company (St. Paul, Minn.).

Crumpability

The ability to be crumpled or crushed into a ball for disposability was measured using the following procedure. A 10 cm by 10 cm sample was cut from a roll of the film and crumpled by hand by rolling between one's fingers and palm to form a tight ball of approximately 1.5 cm diameter. The ball was placed on a flat surface and allowed to relax for 15 seconds. The diameter of the resulting ball was then measured. If the sample did not retain the ball shape and opened up, the observation was recorded as “did not hold ball”.

Coefficient of Friction

The kinetic coefficient of friction (COF) of the films of the invention was measured according to ASTM D1894. The COF was determined for film-to-film contact with the side of the film that contacted the chrome roll being face down on the moving sled and the opposite side of the film being face up on the stationary plate.

Materials Used

PETROTHENE 51S07A: Polypropylene homopolymer, 0.8 g/min MFI (ASTM D1238, 230° C./2.16 kg), (Equistar Chemicals, Houston, Tex.)

White mineral oil #31 (Amoco Oil and Chemical Co., Texas City, Tex.)

MILLAD 3988: Nucleating agent, 3,4-dimethylbenzylidine sorbitol, (Milliken Chemical Co., Inman, S.C.), (available as a 2.5% concentrate in polypropylene as PPA0642495 from Clariant Corp., Minneapolis, Minn.)

PPM71512: Phthalo blue pigment/nucleator concentrate, 80:20 polypropylene:pigment ratio, (Tokyo Printing Ink Co., Tokyo, JP)

POLYWAX 400: synthetic polyethylene wax, 450 MW, 81° C. melting point, (Baker Petrolite, Sugar Land, Tex.)

IGI 1231: refined paraffin wax, 53° C. melting point, (The International Group, Wayne, Pa.)

W-835: microcrystalline wax, 76° C. melting point, (Crompton Corp., Middlebury, Conn.)

### Comparative Example C1

A single layer microporous film was prepared similar to that described in PCT application WO99/29220 Example 1, having the following composition: polypropylene (64%, PETROTHENE 51S07A), mineral oil (35%, white oil #31, Amoco Oil and Chemical Co.) and phthalo blue pigment concentrate (1%). The microporous film had a thickness of approximately 41 microns.

### Comparative Example C2

A single layer microporous film was prepared similar to comparative example C1 except W-835 wax was substituted for the mineral oil. MILLAD 3988 was used at 0.09%. Blue pigment was not used. The microporous film had a thickness of approximately 42 microns.

### Example 1

A three layer A-B-A coextruded microporous film was prepared by using two extruders to provide three melt streams. A 40 mm twin screw extruder was used to supply the B (core) layer consisting of a blend of PETROTHENE 51S07A polypropylene (61.5%), W-835 microcrystalline wax (37.5%) and 1.0% phthalo blue pigment concentrate. The polypropylene was fed into the hopper of a 40 mm twin-screw extruder. The wax solid diluent was melted and pumped through a mass flowmeter and then introduced into the extruder through an injection port at a rate to provide a composition of 61.5% by weight of the polypropylene and 37.5% by weight wax solid diluent. The composition was rapidly heated to 249° C. in the extruder to melt the components after which the temperature was cooled down to and maintained at 193° C. through the remainder of the barrel. The molten composition was pumped from the extruder, through a filter, into a melt pump with a flow rate of 3.6 kg/hr and then via a necktube into an ABA three layer feedblock and then into a coat hanger slit die.

A 25 mm twin screw extruder was used to supply the two A (skin) layers consisting of a blend of PETROTHENE 51S07A polypropylene (65.0%), white #31 mineral oil (35.0%), and 0.065% MILLAD 3988 nucleating agent. The polypropylene was fed into the hopper of a 25 mm twin-screw extruder. The mineral oil liquid diluent was pumped through a volumetric flowmeter and then introduced into the extruder through an injection port at a rate to provide a composition of 65.0% by weight of the polypropylene and 35.0% by weight mineral oil liquid diluent. The composition was rapidly heated to 271° C. in the extruder to melt the components after which the temperature was cooled down to and maintained at 193° C. through the remainder of the barrel. The molten composition was pumped from the extruder, through a filter, into a melt pump with a flow rate of 5.4 kg/hr and then via a necktube into an ABA three layer feedblock and then into a coat hanger slit die. The relative distribution of the three layers was approximately 30/40/30.

The three layer melt curtain was then cast onto a chrome roll (60° C.) running at 6.1 meters/min. The chrome roll had a knurled pattern on it consisting of 40 raised truncated pyramids per centimeter both axially and radially. The cast film was then stretched in-line with a stretching ratio of 1.8 to 1 in the machine direction using a Killion length orienter with the final roll of the preheat section set at 52° C., and a stretching ratio of 1.6 to 1 in the transverse direction using a Cellier tenter having zone temperature settings of 74° C. in all zones to form an opaque light blue microporous film having a thickness of 43 microns, a porosity of 32.9%, a pore size of 0.29 microns and a Gurley airflow of 188 sec/50 cc.

### Example 2

A three layer microporous film was prepared as in Example 1 except IGI 1231 paraffin wax was used as the solid diluent at 35% of the film core layer. Flow rates of 4.5 kg/hr and 5.4 kg/hr were used for the core and skin layers respectively. Phthalo blue pigment concentrate was used at a 1.0% loading in place of the MILLAD 3988 in the skin layers. The relative distribution of the three layers was approximately 27/46/27. The temperature of the chrome roll was maintained at 66° C. A linespeed of approximately 6.9 meters/min was used. The cast film was then stretched in-line with a stretching ratio of 1.8 to 1 in the machine direction using a Killion length orienter with the final roll of the preheat section set at 60° C., and a stretching ratio of 1.7 to 1 in the transverse direction using a Cellier tenter having zone temperature settings of 60° C. in zones 1-6 and 82° C. in heat setting zones 7-8, to form an opaque blue microporous film having a thickness of 43 microns, a porosity of 36.2%, a pore size of 0.11 microns and a Gurley airflow of 849 sec/50 cc.

### Example 3

A three layer microporous film was prepared as in Example 2 except POLYWAX 400 synthetic polyethylene wax was used as the solid diluent at 35% of the film core layer. Flow rates of 3.6 kg/hr and 5.4 kg/hr were used for the core and skin layers respectively. The relative distribution of the three layers was approximately 27/46/27.The temperature of the chrome roll was maintained at 66° C. A linespeed of approximately 6.0 meters/min was used. The cast film was then stretched in-line with a stretching ratio of 1.8 to 1 in the machine direction using a Killion length orienter with the final roll of the preheat section set at 77° C., and a stretching ratio of 1.7 to 1 in the transverse direction using a Cellier tenter having zone temperature settings of 74° C. for all zones to form an opaque blue microporous film having a thickness of 43 microns, a porosity of 36.8%, a pore size of 0.12 microns and a Gurley airflow of 238 sec/50 cc.

Table 1 below shows that crumpable films with low coefficient of friction surfaces can be obtained by coextruding microporous skin layers containing liquid diluent with a microporous core layer made from a solid diluent.

TABLE 1

| | Crumpability | Coefficient | Porosity | Gurley Air | Pore Size |
|---|---|---|---|---|---|
| Example | (cm) | of Friction | (%) | Flow (sec) | (microns) |
| C1 | 4.1 | 0.686 | 42.3 | 36 | 0.41 |
| C2 | 1.9 | 1.013 | 34.6 | 130 | 0.16 |
| 1 | 2.5 | 0.580 | 32.9 | 188 | 0.29 |
| 2 | 2.4 | 0.484 | 36.2 | 849 | 0.11 |
| 3 | 1.7 | 0.431 | 36.8 | 238 | 0.12 |

We claim:

1. An oil absorbing wipe suitable for wiping a users skin or hair comprising an oil absorbing porous coextruded film substrate having a crumpable layer of a crystalline thermoplastic material, a matrix of thermoplastic domains interconnected by fibrils; and solid diluent present between the domains, the solid diluent being miscible with the thermoplastic and at least one second outer microporous layer of a thermoplastic material and a diluent which wipe changes transparency or color when loaded with oil, the wipe having the ability to crumple to 3.0 cm or less, for a 10 by 10 cm wipe.

2. The oil absorbing wipe of claim 1, wherein the solid diluent is a wax.

3. The oil absorbing wipe of claim 2, wherein the solid wax is at least one of paraffin wax, microcrystalline wax, and polyethylene wax.

4. The oil absorbing wipe of claim 1, wherein the solid diluent is a polymer or copolymer.

5. The oil absorbing wipe of claim 1, wherein the thermoplastic material for both layers is a polyolefin.

6. The oil absorbing wipe of claim 1, wherein the solid diluent at least partially surrounds the polyolefin domains in the crumpable layer.

7. The oil absorbing wipe of claim 5 wherein the polyolefin is at least one of polyethylene, polypropylene, polybutenes, polyisoprene, polymethylpentene, and copolymers thereof.

8. The oil absorbing wipe of claim 5 the crumpable layer comprising:
(a) 25 to 75 wt-% polyolefin; and
(b) 25 to 75 wt-% solid diluent.

9. The oil absorbing wipe of claim 8 the crumpable layer comprising:
(a) 25 to 50 wt-% high-density polyethylene; and
(b) 50 to 75 wt-% solid diluent.

10. The oil absorbing wipe of claim 9 the crumpable layer comprising at least 55 wt-% polyethylene wax, microcrystalline wax, or paraffin wax.

11. The oil absorbing wipe of claim 8 the crumpable layer comprising:
(a) 30 to 75 wt-% polypropylene; and
(b) 25 to 70 wt-% solid diluent.

12. The oil absorbing wipe of claim 11 the crumpable layer comprising about 35 to 40 wt-% polyethylene wax.

13. The oil absorbing wipe of claim 11 the crumpable layer comprising about 35 to 70 wt-% paraffin wax or microcrystalline wax.

14. The oil absorbing wipe of claim 8 the crumpable layer comprising:
(a) 35 to 55 wt-% methylpentene copolymer; and
(b) 45 to 65 wt-% solid diluent.

15. The oil absorbing wipe of claim 14 the crumpable layer comprising at least 45 wt-% paraffin wax.

16. The oil absorbing wipe of claim 15 the crumpable layer comprising 50 to 55 wt-% paraffin wax.

17. The oil absorbing wipe of claim 1 wherein the diluent in the second microporous layer is a liquid hydrocarbon.

18. The oil absorbing wipes of claim 17 wherein the diluent is a lower molecular weight hydrocarbon.

19. The oil absorbing wipes of claim 17 wherein the diluent is a higher molecular weight liquid hydrocarbon.

20. The oil absorbing wipe of claim 1 wherein interstitial volume per unit area of said microporous stretched film is in the range of 0.0001-0.005 cm³ as calculated by the following equation:
interstitial volume per unit area=[film thickness (cm)×1 (cm)×void content (%)]/100 (where the void content is the percentage of voids in the porous film).

21. The oil absorbing wipe of claim 20 wherein the porous film voids have an average size in the range of from 0.1 to 5.0 microns (μm).

22. The oil absorbing wipe of claim 20 wherein the interstitial volume per unit area is from 0.0002 to 0.001 cm³.

23. The oil absorbing wipe of claim 1 wherein the crumpable film layer has a thickness of at least 10 microns.

24. The oil absorbing wipe of claim 23 wherein the crumpable film layer has a thickness of at least 10 to 25 microns.

25. The oil absorbing wipe of claim 23 wherein the crumpable film layer has a thickness of at least 15 to 20 microns.

26. The oil absorbing wipe of claim 1 wherein the wipe coextruded film layers has a thickness of at least 20 microns.

27. The oil absorbing wipe of claim 26 wherein the wipe coextruded film layers has a thickness of at least 35 to 45 microns.

28. The oil absorbing wipe of claim 1 wherein the at least second microporous film layer has a thickness of at least 5 microns.

29. The oil absorbing wipe of claim 26 wherein the second outer microporous layer has a thickness of at least 5 to 15 microns.

30. The oil absorbing wipe of claim 1 wherein the wipe has a Coefficient of Friction on at least one face of less than 0.75.

31. The oil absorbing wipe of claim 1 wherein the wipe has a Coefficient of Friction on at least one face of less than 0.60.

32. A package of oil absorbing wipes comprising two or more wipes of claim 1 in a package.
